(19) United States
(12) Patent Application Publication
(10) Pub. No.: US 2007/0299334 A1
(43) Pub. Date: Dec. 27, 2007

(54) MEDICAL INSTRUMENT WITH A TOUCH-SENSITIVE TIP AND LIGHT EMISSION SOURCE

(76) Inventor: Stefan Vilsmeier, Munchen (DE)

Correspondence Address: DON W. BULSON (BrainLAB), RENNER, OTTO, BOISSELLE & SKLAR, LLP, 1621 EUCLID AVENUE - 19TH FLOOR, CLEVELAND, OH, 44115, US

(21) Appl. No.: 11/763,462

(22) Filed: Jun. 15, 2007

Related U.S. Application Data
(60) Provisional application No. 60/804,945, filed on Jun. 16, 2006.

(51) Int. Cl. A61B 19/00

(52) U.S. Cl. 600/414

(57) ABSTRACT

A medical instrument for registering or referencing a body or body part in a medical navigation system includes a body, a tracking device trackable by the navigation system, an instrument tip having a touch sensor, and an electromagnetic radiation source for producing a light beam. The touch sensor detects contact between the tip and an object, and via the tracking device, the location of the tip can be ascertained at the moment contact between the tip and the object is made. Additionally, the light beam can be projected onto a surface of the body or body part to create a plurality of markers that also are trackable by the navigation system.

### RELATED APPLICATION DATA

This application claims priority of U.S. Provisional Application No. 60/804,945 filed on Jun. 16, 2006, which is incorporated herein by reference in its entirety.

### FIELD OF THE INVENTION

The present invention relates to a method and apparatus for referencing and/or registering a patient or a patient body part in a medical navigation system.

### BACKGROUND OF THE INVENTION

Surgical operations or radiotherapy are increasingly being performed with the aid of so-called navigation or tracking systems. In such systems, patient data determined by an imaging technique, such as a computer tomography or magnetic nuclear resonance tomography, for example, is used to indicate to the treating doctor a location of a surgical instrument relative to the patient's body or body part.

To enable such navigation systems, the momentary location of the patient, the part of the body to be treated and/or a surgical instrument is identified or “referenced” in three dimensional space to the navigation system. Once identified, the navigation system correlates or “registers” the position of the body to the previously obtained patient data. Further, via reference markers or the like, the navigation system tracks movement of the body and/or surgical instruments in three-dimensional space. The navigation system then displays the location of the surgical instruments relative to the body and/or medical data on a display, for example.

Conventionally, markers are applied to the skin surface of the patient prior to obtaining the patient data. The employment of such externally applied markers to the surface of the skin harbors several disadvantages. For example, the skin surface is easily shiftable, and such shifts result in inaccuracies during referencing and subsequent registration. In particular, when targeting the markers with a pointer tool, the skin may be slightly displaced. Further, the accuracy of the registration is related to the number of markers on the patient. The higher the number of markers, the higher the degree of accuracy in the registration. However, as the number of markers are increased, the time required to reference all the markers also is increased, as each marker must be identified to the navigation system. Thus, there is a tradeoff between accuracy and registration time.

Invasive solutions such as, for example, attaching the markers to the bone substance under the skin, are unpleasant for the patient, while natural landmarks such as the root of the nose, for example, often cannot be referenced with good positional accuracy.

Another disadvantage of the above-cited technique is apparent when the treatment is not performed directly after tomography. Thus, for instance, some of the markers may become detached overnight, which can lead to serious difficulties during referencing. One particularly disadvantageous case can occur when the detached markers are replaced by the patient himself at some other location, which may result in erroneous treatment.

Other methods of registration also are known, such as is disclosed in U.S. patent application Ser. No. 10/134,974, for example, which describes a registration method and device using a medical instrument having a touch-sensitive tip. The device can be tracked by a medical navigation system such that the location of the tip in three-dimensional space is known. Points on the patient may be identified or referenced to the navigation system by touching said points with the tip of the instrument. The touch-sensitive tip identifies the moment contact is made with the surface, and the instrument transmits a signal to the navigation system indicative of said contact. Since the navigation system knows the location of the instrument tip, the navigation system also knows the location of the identified point.

A disadvantage of the above method is that while the instrument tip is touch sensitive, in certain cases there still may be some deformation at the point as the tip is placed on the point. For example, when identifying locations on the patient having large amounts of fatty tissue and/or skin, the tip of the instrument may slightly push into or deform the tissue at the instant contact is made and/or while the navigation system calculates the location of the tip in three dimensional space, resulting in erroneous data.

U.S. Pat. No. 6,873,867 discloses a registration method and device that emits or beams a light source on the surface of the patient, thereby creating points on the surface. A medical navigation system can identify the location of the points in three-dimensional space and register the patient to previously obtained medical images or data. Since the light beam does not deform the surface of the patient, errors are minimized. However, registration using the light beam requires that the beam actually strike the intended surface. If the surface has large amounts of hair, for example, then points may be generated away from the actual surface, thereby creating erroneous data. Thus, it becomes necessary to remove any hair in the area that is being registered or to employ a different registration technique.

### SUMMARY OF THE INVENTION

According to the present invention, there is provided a medical instrument for referencing points on a patient or a patient body part during registration in a medical navigation system, such as an optical or magnetic navigation system, for example. More particularly, the instrument can be used to implement multiple methods of referencing a patient or patient body part.

The medical instrument can include an instrument body having an instrument tip, and a radiation source for emitting electromagnetic radiation in the infrared, visible and/or ultraviolet spectrum, for example. A tracking device, such as a marker or reference star, can be attached to the instrument and used by the medical navigation system to identify the location of the instrument in three-dimensional space. The instrument tip can include a touch sensor that detects when the instrument tip touches an object, e.g., a surface of the patient's skin. Further, the electromagnetic radiation source can project light markers on the surface of the patient or body part, wherein the light markers can be detected by a camera system or the like. As is described in more detail below, both the instrument tip and the emitted electromagnetic radiation can be used to register the patient or patient body part.

Accordingly, a medical instrument for registering a body or body part in a medical navigation system comprises a body, an instrument tip including a touch sensor, wherein the touch sensor detects contact between the tip and an object, a tracking device trackable by the navigation system, and a first electromagnetic radiation source for producing a first light beam on a surface of the body or body part to produce a plurality of markers, wherein the markers are trackable by the navigation system.

The forgoing and other embodiments of the invention are hereinafter discussed with reference to the drawings.

### BRIEF DESCRIPTION OF THE DRAWINGS

FIG. 1 illustrates an exemplary medical instrument for referencing and registering a patient in accordance with the invention.

FIG. 2 illustrates the instrument of FIG. 1 referencing a patient via light markers generated on a surface of the patient.

### DETAILED DESCRIPTION

A trackable medical instrument for registering a patient is provided, wherein the instrument includes a touch sensitive tip and an electromagnetic radiation source that produces electromagnetic radiation in the infrared, visible and/or ultraviolet spectrum (e.g., an infrared light source). Both the touch sensitive tip and the electromagnetic radiation source can be used to reference and register a patient or a body part of the patient in a medical navigation system or the like. Moreover, each referencing method (e.g., touch and light) can be used independent of the other or in combination, to verify the accuracy of the respective referencing techniques.

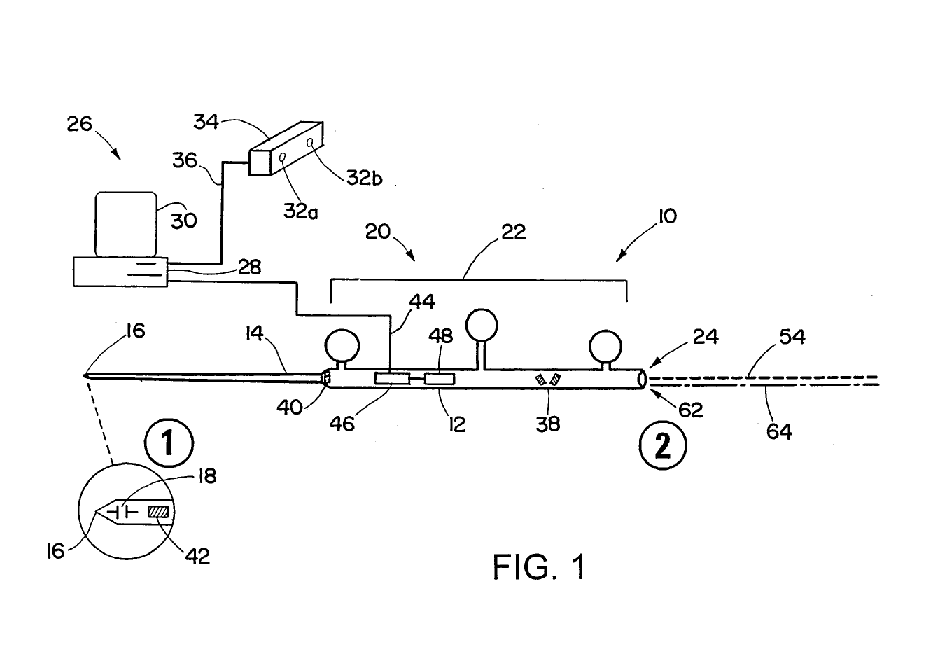

Referring to FIG. 1, there is provided a medical instrument 10 which includes a body 12 coupled to a front extension 14 and a tip 16. The tip 16 is fitted with a touch sensor 18, such as a capacitive sensor or the like, as can be seen in the enlarged view of the tip 16. Also coupled to the body 12 is a tracking device 20, such as one or more markers arranged in a predetermined configuration to form a marker array 22 (e.g., a reference star). Alternatively, the tracking device 20 can include a plurality of induction coils as discussed in more detail below. The coils can be used with magnetic navigation systems, while the marker array 22 can be used with optical navigation systems. The tracking device 20 also can include a combination of optical and magnetic tracking devices, thus enabling the instrument 10 to be used with both optical and magnetic navigation systems. As will be described in more detail below, the instrument 10 also includes an electromagnetic radiation source 24, such as an infrared light source or the like, for emitting a light beam on a surface of the patient or patient's body part.

FIG. 1 also illustrates an exemplary optical navigation system 26, which includes a computer 28, display 30 and cameras 32a and 32b. The computer 28 may be coupled to a camera mount 34 via a cable connection 36, and the two infrared cameras 32a and 32b, which monitor a target area, are attached to the camera mount 34 and spaced apart from each other. The computer 28 can be communicatively linked to the cameras 32a and 32b via the cable connection 36. As will be appreciated, other types of navigation systems may be employed in place of the optical navigation system 26.

The marker array 22 can include active and/or passive markers, which are detectable by the cameras 32a and 32b of the medical navigation system 26. Active markers emit radiation, such as infrared light, for example, while passive markers reflect radiation from another source, e.g., from the cameras. Instruments configured for optical navigation systems include at least two and preferably three markers arranged at a known orientation on the instrument 10.

As was discussed above, the instrument 10 can include coils for use with magnetic navigation systems. The coils can be provided at a number of locations on the instrument 10, such as, for example, a pair of coils 38 in the instrument body 12, a single coil 40 at a transition point between the instrument body 12 and the extension 14, and a single coil 42 directly in the tip 16. The pair of coils 38 form a so-called “six-dimensional” sensor for magnetic tracking, while the coils 40 and 42 each form a “five-dimensional” magnetic tracking sensor, and together a “six-dimensional” magnetic tracking sensor. The pair of coils 40 and 42 alone suffice as a sensor unit, as does the pair of coils 38.

A cable 44 coupled to a transmitter 46 can transmit a signal to the navigation system 26 when the tactile tip 16 reports a contact. The transmitter 46 can be any conventional transmitter used for transmitting analog and/or digital signals. Though not shown, it is conceivable to transmit the data to the navigation system 26 via a wireless communication link or the like.

As noted above, the tip 16 includes a touch-sensitive sensor 18, which may be any touch-sensitive sensor 18 known in the technical field. Depending on the application, the touch-sensitive sensor can be an electrical resistance sensor, a capacitance sensor or a mechanical sensor, for example. The touch sensor 18 can be selected such that a signal is generated only for particular object surfaces, thereby minimizing the likelihood of false signals. Thus, the tip 16 operates as a small, sensitive switch or electronic sensor capable of detecting surface contact, and can function mechanically, electrically and/or magnetically. Each time the tip 16 touches the surface of the object, a signal (which can be digital or analog) is generated or set.

The touch sensitive tip 16 can be used for highly accurate contour detection on the surface of the object to be registered, since a point can be referenced before the surface of the skin is pressed or indented. A surface of the object and its spatial position can be determined from the spatial position of the instrument 10, which is obtained by the navigation system 26 via the tracking device 20 (the arrangement of coils and/or markers). No further manual switches are necessary. When a sufficient number of points on the surface have been referenced by contact, surface-matching allows registration without markers, using previously obtained images. For further details regarding a touch sensor and its implementation, reference may be had to U.S. Published application No. 2003-0069588 A1 which is hereby incorporated herein in its entirety.

To avoid or minimize the likelihood of the touch sensitive tip 16 generating signals constantly or in quick succession, a control system 48 can be assigned to the transmitter 46, wherein the control system 48 only allows the transmitter 46 to transmit another signal after a predetermined period of time has elapsed (e.g., one transmission per three seconds). Alternatively, the control system 48 may be configured as a one-shot, wherein the transmitter is permitted to transmit a signal only at the instant the tip touches the object. Further transmissions would require that the tip 16 has at least been removed from the surface for a predetermined time. Using the signal transmission control system 48 incorporating a delay, filter, or one-shot scheme, referencing signals are inhibited while the surface of the skin is pressed in, thereby minimizing the transmission of false signals.

The tip 16 may be removed from the instrument body 12 and/or extension 14 and individually sterilized or kept sterile. Additionally, the tip 16 can be disposable and packaged sterile. Thus, further sterilization of the tip 16 would not be required.

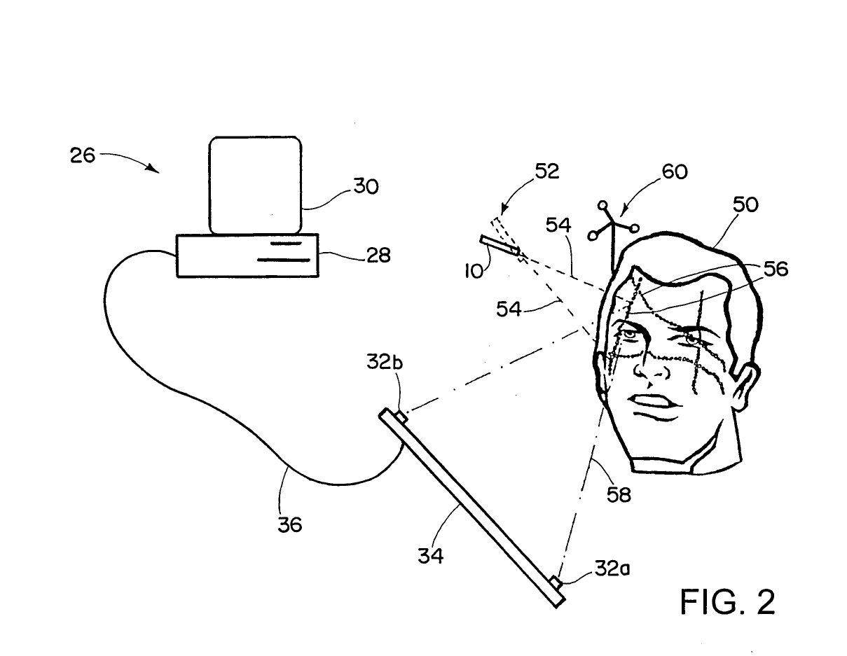

As was noted above, the instrument 10 also includes an electromagnetic radiation source 24, such as an infrared laser light source, for example. Like the touch-sensitive tip 16, the electromagnetic radiation source 24 also can be used to reference and register the patient or patient's body part. For example, and with further reference to FIG. 2, the position of a human head 50 is to be registered in the navigation system 26. The instrument is held such that the electromagnetic radiation source 24 emits a light beam 54, wherein the instrument 10 is shown by the broken line in a second position to simulate a swinging motion of the instrument, thereby scanning the facial surface of the patient's head 50. The facial surface is scanned by the referencing light beam 54 for a period of time, resulting in the production of sequential light reflections or light markers 56 (e.g., a cloud of light spots) being produced on the facial surface. While only a few such light markers 56 are represented in FIG. 2, it will be appreciated that a number of lights markers 56 are projected on the surface. Alternatively, the light markers 56 can be projected at individual locations (as opposed to using a swinging or scanning motion). In other words, the light beam 54 can be projected on a single location to create a light marker 56, and then the light beam can be turned off (e.g., via a switch on the body) while the instrument 10 is aimed at a new location. Once aimed, the light beam 54 can be enabled to produce a light marker 56 at the new location. This can be repeated until a sufficient number of points have been collected. Theoretically, detecting just three points is sufficient. However, accuracy is increased when more points are generated. A sufficient number of light markers 56 provides enough information to enable one to three-dimensionally assign the surface with high accuracy. Tests have shown that a low number (roughly 20) of light markers 56 is sufficient to determine the spatial location of the body part with good accuracy.

Due to the fast recording in sequence of single images, the cameras 32a and 32b capture respective light reflections 58 each arranged in sequence, wherein the light path for a single light marker 56 is indicated by the dot-dash line. The two cameras 32a and 32b three-dimensionally map the spatial location of the light markers 56 and the computer system 28 determines from the camera data the position of the light markers 56 on the facial surface.

Stored in the computer 28 are previously obtained data from a scan of the patient's head, and thus also the data for the facial surface. The computer 28 then executes a matching routine to determine whether the number of surface points obtained from the light markers 56 is sufficient to assign or make congruent the detected surface points of the surface with the data from the scan data set. Once sufficient agreement exists, an acoustic and/or visual signal is output to indicate to the person conducting treatment that referencing has been successfully concluded.

Accordingly, the electromagnetic radiation source 24 produces light markers 56 on the target surface simply by beaming light on the surface (of the skin, bone) to effectively produce markers on the surface. Preferably, the light is invisible to the human eye (e.g., infrared light) but visible to the cameras 32a and 32b as the reflection of a marker. The light markers 56 generated thus eliminate the need for attached markers or markers otherwise applied to the patient. Moreover, the plurality of light markers 56 enable the navigation system 26 to reference and register the patient or body part with a high degree of accuracy.

Also schematically shown in FIG. 2 is reference adapter 60 fixedly positioned to the head 50 of the patient. This adapter 60 includes three markers (active and/or passive), the positions of which can be tracked by the cameras 32a and 32b. Should it be necessary to turn the head 50 of the patient during referencing or to move the cameras 32a and 32b to eliminate camera shadows, for instance by the nose, the relative movement is tracked with the aid of the adapter 60 and taken into account in referencing such that errors are avoided.

It is advantageous to create clearly discernible light spots on the surface of the patient body part as the light markers. Thus, the beam should be directed as much as possible at an impact location. In this respect, it is advantageous to use a beam of invisible light, since the light marker 56 produced thereby can be clearly distinguished from the light reflections generated by room lighting otherwise illuminating the patient body part. Employing an infrared light beam is of advantage in this respect, as the cameras can be set to detect infrared reflections. Very well defined and directed light spots can be obtained by using laser light.

When, as proposed above, invisible light is employed to generate the light marks, it is in principal difficult to establish where the light beam is being beamed at any one time. To solve this problem, a visible light reflection is generated on the surface by means of a second beam of visible light, aimed substantially at the same target area as the invisible referencing light beam. Thus, the instrument 10, in addition to the electromagnetic radiation source 24 that creates the invisible light beam 54, also can include a visible light source 62 that projects a visible light beam 64 in the same direction and with the same focus to enable the person conducting treatment to visually track the light markers 56 generated by the electromagnetic radiation source 24 and to prevent beaming into the eyes. The two light sources can be located juxtaposed or nested, for example, or a single light source that generates both visible and invisible light can be used.

With the aid of the second light beam 64, it is possible to clearly identify the location of the invisible referencing light beam 54. This second light beam 64 may also be a laser light beam. Use of the visible light beam 64 can avoid potential danger or harm to the patient. For example, since the light beam 54 is invisible to the human eye and accidental beaming into the open eye produces no eyelid blink reflex, there is a risk of injury, such as retinal burns. However, when a second beam of visible light is used, the visible light beam can serve as a target aid, excluding sensitive areas (e.g., the eyes) from electromagnetic radiation. Further, the visible light prompts eyelid blink reflex when entering the eye to thus prevent corneal burns.

Further details of light beam registration may be had by reference to U.S. Pat. No. 6,873,867 which is hereby incorporated herein by reference.

An advantage of the instrument 10 is that it can be used to register a patient via multiple referencing techniques. Thus, the surgeon need not switch instruments when a particular technique is not suitable for the area to be registered (e.g., excessive hair or fatty tissue may dictate that a particular method is not preferred). Further, one technique may be employed to reference and register the patient, and the other technique may be employed to verify or check the accuracy of the overall registration.

Another advantage of using the instrument 10, in addition to the high registration accuracy, is that it eliminates the problems associated with the use of separately applied markers. Referencing does not suffer from markers being displaced, shifted or removed. Further, unpleasant side-effects relating to separately applied markers, such as the distress of markers being affixed or even invasively secured in place, are eliminated.

Another benefit of registration via the instrument 10 is that separate markers need not be attached and, therefore, surgery can be decoupled in time from the tomographic scan. Since there is no substantial change in the skin surface of the patient over lengthy periods, it is now possible to precisely reference the patient several days after the tomographic scan without the patient retaining markers on the skin surface over a long period of time.

Further, registration via the instrument 10 enables the surgeon to employ any number of scans for navigational purposes. Thus, should a physician establish prior to the operation that a further CT scan done some weeks beforehand would be of help during navigation, it now poses no problem for him to also employ this during navigation, since it is not necessary that markers are imaged in such a scan.

Additionally, an already existing camera system can be used with both the touch-sensitive and light emitting features of the instrument 10. Thus, additional devices, such as laser scanners, are not needed. Compared to conventional methods, the present method permits enhanced accuracy and faster detection.

The referencing system may be employed with all methods of treatment involving an image-assisted operation. This applies to both surgical operations and radiation treatments. Referencing can also be employed for tracking systems with passive marker arrays as well as for those with active emitter markers, as used for instance in tracking medical instruments. Although hitherto it has mainly been indicated that the light markers are generated by means of the light beam on the skin surface of the patient, it is also conceivable to reference bone structures already exposed for treatment, for instance, exposed bone portions of the skull or spine.

Referencing can be employed for any surgery requiring the momentary three-dimensional position of a patient body part to be determined. The invention, however, is suitable for methods in which the three-dimensional position of the surface is assigned to a set of image data, previously produced by an imaging technique, for the body part, especially in a CT, MRI (magnetic nuclear resonance tomograph). PET, SPECT, x-ray or ultrasound scan data set to update-reference the image data of this data set.

Although the invention has been shown and described with respect to a certain preferred embodiment or embodiments, it is obvious that equivalent alterations and modifications will occur to others skilled in the art upon the reading and understanding of this specification and the annexed drawings. In particular regard to the various functions performed by the above described elements (components, assemblies, devices, compositions, etc.), the terms (including a reference to a “means”) used to describe such elements are intended to correspond, unless otherwise indicated, to any element which performs the specified function of the described element (i.e., that is functionally equivalent), even though not structurally equivalent to the disclosed structure which performs the function in the herein illustrated exemplary embodiment or embodiments of the invention. In addition, while a particular feature of the invention may have been described above with respect to only one or more of several illustrated embodiments, such feature may be combined with one or more other features of the other embodiments, as may be desired and advantageous for any given or particular application.

What is claimed is:

1. A medical instrument for registering and/or referencing a body or body part in a medical navigation system, comprising:
an instrument body;
an instrument tip coupled to said body and including a touch sensor, wherein the touch sensor detects contact between the tip and an object;
a tracking device coupled to said body and trackable by the navigation system; and
a first electromagnetic radiation source for producing a first light beam on a surface of the body or body part to produce a plurality of markers, said markers being trackable by the navigation system.

2. The instrument of claim 1, wherein the first light beam is an invisible light beam.

3. The instrument of claim 2, wherein the first light beam is an infrared light beam.

4. The instrument of claim 2, wherein the first electromagnetic radiation source produces a second light beam, said second light beam being a visible light beam aimed at substantially the same target area as the first light beam, whereby a visible light reflection can be generated on the body or body part.

5. The instrument of claim 1, further comprising a second electromagnetic radiation source producing a second light beam, said second light beam being a visible light beam aimed at substantially the same target area as the first light beam, whereby a visible light reflection can be generated on the body or body part.

6. The instrument of claim 5, wherein the first and second electromagnetic radiation sources are two juxtaposed or nested light sources.

7. The instrument of claim 5, wherein the first and second electromagnetic radiation sources are laser light sources.

8. The instrument of claim 1, wherein the first electromagnetic radiation source is a laser light source.

9. The instrument of claim 1, wherein the tracking device is an optical marker array.

10. The instrument of claim 9, wherein the optical marker array includes active and/or passive markers.

11. The instrument of claim 1, wherein the tracking device is a magnetic tracking device.

12. The instrument of claim 11, wherein the magnetic tracking device is at least one magnetic coil.

13. The instrument of claim 1, further comprising a transmission device that transmits a signal when a contact is detected.

14. The instrument of claim 13, further comprising a control system assigned to the transmission device and/or sensor, said control system only allowing the transmission device and/or sensor to transmit a signal based on a predetermined criteria.

15. The instrument of claim 14, wherein the predetermined criteria is a one-shot or based on a predetermined period of time.

16. The instrument of claim 1, wherein the touch sensor is an electrical resistance sensor, a capacitance sensor, a mechanical sensor or a combination of said sensors.

17. The instrument of claim 1 wherein said tip is removable from said body.

18. A system for referencing or registering a body or body part in a medical navigation system, comprising:
the medical instrument as set forth in claim 1;
a plurality of cameras configured to detect optical data on the surface of the body or body part generated by the first electromagnetic radiation source,
said cameras also configured to detect optical data generated or reflected by the tracking device, wherein the plurality of cameras provide positional data related to respective locations of the optical data; and
a processor operatively coupled to the plurality of cameras for receiving the positional data from the cameras and executing program code to determine a three-dimensional position in space for optical data, and to reference or register the body or body part based on the three dimensional position of the optical data.

19. A method of registering a patient or a patient body part in a medical navigation system, comprising:
using the instrument of claim 1 to identify a first set of points on the patient or patient body part to a medical navigation system, said identification of the first set of points including contacting a surface of the patient or patient body part with the touch sensitive tip;
using the instrument of claim 1 to identify a second set of points on the patient or patient body part to the medical navigation system, said identification of the second set of points including beaming the light beam on the surface of the patient or patient body part; and
using one of the first set of points or the second set of points to register the patient or patient body part to previously obtained tomographic data of the patient or patient body part, and using the other of the first set of points or the second set of points verifying said registration.
